SEQUENCE LISTING (1 sequence)

SEQ ID NO: 1 - DNA, 1363 bp, Pichia pastoris
ccgctcgagaaaagagaggctgaagctgttgcctgcaaggatgagatcacacaaatgacctacagaagacaggagtcttggttgagacctgaagtcagatctaagagagttgagcactgccagtgtgacagaggtcaagcaagatgtcataccgttcctgttaaatcttgttctgagccaagatgcttcaatggaggaacctgccaacaagccttgtacttttctgatttcgtctgccaatgcccagagggtttcgccggtaagtgttgcgagatcgacaccagagctacatgctatgaggatcagggtatctcttatagaggaacttggtctacagccgagtctggtgctgaatgtacaaattggaactcttctgccttggcccaaaagccatactctggtagaagacctgatgccattagattgggattgggtaaccacaactattgtagaaacccagatagagactctaagccatggtgctacgtctttaaggccggaaagtattcttctgagttttgttctaccccagcttgctctgagggtaactctgactctacctgcggattgagacagtactctcagcctcagtttagaattaagggtggattgtttgcagacatcgcctctcacccatggcaggctgcaatttttgctgctgccgcagcctctccaggagagagatttttgtgcggtggtatcttgatttcttcttgctggattttgtctgccgctcactgttttcaggaaagatttccacctcatcatttgacagtcattttgggaagaacttacagagttgttccaggtgaagaggagcagaagtttgaggtcgagaaatacatcgttcacaaggaatttgatgatgacacatacgacaacgacattgcattgttgcagttgaaatctgattcttccagatgtgcccaggaatcttctgttgttagaactgtctgcttgcctccagcagatttgcaattgccagattggaccgaatgcgaattgtctggttatggtaagcacgaggcattgtctcctttttactctgaaagattgaaggaggcccatgtcagattgtacccatcttccagatgcacttctcagcacttgttgaatagaacagttaccgacaatatgttgtgtgctggtgacacaagatctggtggtccacaagccaacttgcatgacgcttgccaaggtgattctggaggtcctttggtttgtttgaatgacggaagaatgactttggtcggaatcatctcttggggattgggttgcggacaaaaggatgtcccaggagtttatacaaaagtcacaaactatttggactggatcagagataacatgagaccagctctagagc

(19) United States
(12) Patent Application Publication
(10) Pub. No.: US 2015/0259666 A1
(43) Pub. Date: Sep. 17, 2015

(54) CHIMERIC TISSUE PLASMINOGEN ACTIVATOR (T-PA) RESIATANT TO PLASMINOGEN ACTIVATOR INHIBITOR-1 AND IMPROVED BIOCHEMICAL PROPERTIES

(71) Applicant: Pasteur Institute of Iran (IPI), Tehran (IR)

(72) Inventors: MohammadReza Kazemali, Tehran (IR); Amir Hossein Saadati, Tehran (IR); Keivan Keivan Majidzadeh, Tehran (IR); Soroush Soroush Sardari, Tehran (IR); Fatemeh Davami, Tehran (IR); Fereidoun Mahboudi, Tehran (IR)

(21) Appl. No.: 14/672,213

(22) Filed: Mar. 29, 2015

(51) Int. Cl. C12N 9/72; C07K 14/485

(52) U.S. Cl. CPC C12N 9/6459; C07K 14/485; C07K 2319/00

(57) ABSTRACT

The present invention discloses the thrombolytic therapy by t-PA or CT-b for the treatment of the acute myocardial infarction. A chimeric truncated form of t-PA or CT-b is designed and expressed in Pichia pastoris. The CT-b includes desmoteplase finger domain, human EGF, kringle 1 and protease domain. The human kringle 2 domain is removed in CT-b to make it structurally and functionally similar to desmoteplase. The fibrin specificity or the catalytic activity is 1560 times more in the presence of fibrin. The CT-b also shows 1.2 fold higher resistances to PAI-1 enzyme. As the kringle domain is considered as one of the binding sites for PAI-1, the deletion along with amino acid substitution in protease domain contributes to prolonged half-life. Further the activity of the CT-b is intact after exposure to PAI-1. In other words CT-b is inhibited 44% less than t-PA by PAI-1 enzyme, demonstrating improved half life.

### BACKGROUND

1. Technical Field

The embodiments herein generally relate to the field of bio-engineering of drugs by recombinant technology. The embodiment herein particularly relate to the synthesis of thrombolytic drugs and particularly to tissue plasminogen activators (t-PA). The embodiments herein more particularly relate to a novel variant of tissue plasminogen activator (t-PA) with improved pharmacodynamic properties compared to native tissue plasminogen activator.

2. Description of the Related Art

Cardiovascular disease especially heart stroke is one of the main reasons of morbidity and mortality in the word. One of the conventional drug groups used from past years are thrombolytic drugs including plasminogen activator.

Coronary heart disease (CHD) is the most common form of heart and cardiovascular diseases. Acute ischemic stroke (AIS) is the most common cause of death which can be classified into different categories according to the presumed mechanism. Cardioembolic strokes present a major form of ischemic strokes and acute myocardial infarction (AMI). The acute myocardial infarction (AMI) is a cardiac condition that has been associated with cardioembolic strokes. AMI is commonly caused by atherosclerotic occlusion of the coronary arteries, which is the result of a thrombus or clot forming on top of a ruptured atherosclerotic plaque, blocking the blood flow through the artery.

Recognition of the importance of fibrinolytic system in thrombus resolution has resulted in the development of various fibrinolytic agents and plasminogen activators (PAs) with different pharmacokinetic and pharmacodynamic properties.

Plasminogen activators are of great clinical significance as thrombolytic agents for management of stroke and myocardial infarction. Fibrinolysis therapy has been subject of interest from many years. Fibrinolysis therapy eliminates many of the associated side effects of the conventional fibrinolytic drugs. Further new generations of fibrinolytic drugs are associated with limitations that their usage in clinical practice remains challenging. The human tissue plasminogen activator (t-PA) is exploited in acute myocardial infarction is used vastly in emergency wards. This protein includes five domains. The five domains are finger domains, epidermal growth factor (EGF), kringle 2, kringle 2 and protease domain. When a clot is formed in heart, lung or other deep veins, the t-PA is naturally produced from epithelial cells and in combination with plasminogen and fibrin t-PA forms a tertiary complex accelerating further production of t-PA.

Tissue-type plasminogen activator (t-PA) is generally preferred for its more efficacy and safety compared to urokinase and streptokinase. Tissue-type plasminogen activator (t-PA) is a glycoprotein consisting of 527 amino acid residues (72 KDa) with seventeen disulfide bonds and approximate 7% carbohydrate in total molecular weight. The t-PA has an enhanced activity in the presence of fibrin, i.e. fibrin-specific plasminogen activation is the major advantage of t-PA over other thrombolytic agents. The tissue-type plasminogen activator (t-PA) is mainly released by endothelial cells. The t-PA cleaves the zymogen plasminogen into active plasmin. Further the plasmin degrades fibrin, as the major component of clots, and promotes blood reperfusion. The type-1 plasminogen-activator inhibitor (PAI-1) and a2-antiplasmin (a2-AP) inhibit this cascade by blocking the proteolytic activity of t-PA and plasmin, respectively. The PAI-1 belongs to serpin family which plays its role as an ideal pseudo-substrate for target serine proteases. The first source of PAI-1 is synthesized by endothelial cells and/or by hepatocytes. The second pool of PAI-1 is contained within the a-granules of platelets. The interaction between t-PA and PAI-1 bound to fibrin is composed of three sequential steps: (a) interaction of the catalytic site of t-PA with the reactive center of PAI-1, bound to fibrin, (b) conformational change in the complex that leads to loss of t-PA's affinity for fibrin, and (c) dissociation of t-PA from the fibrin matrix and rebinding to fibrin subsequently; that would greatly impede t-PA activity.

Tissue-type plasminogen activator (t-PA) is the dominant PA involved in fibrinolysis. The t-PA is a glycoprotein with 67 kDa, 527 amino acids, which promotes conversion of plasminogen to plasmin in the presence of fibrin. The protein molecule is divided into five structural domains: finger domain (F) followed by a growth factor domain (EGF) near the N-terminal region and the two kringle 1 (K1) and kringle 2 (K2) domains. Next to kringle 2 domain is the serine protease domain with the catalytic site at the C terminus. Both finger and kringle 2 bind to the fibrin and accelerate t-PA activation on plasminogen. However, full length t-PA has some major disadvantages i.e. the rapid clearance from plasma due to the recognition of structural elements on first three N-terminal domains by certain hepatic receptors is the most important. Human fibrinogen can be converted to fibrin through thrombin catalyzed release of small peptides from the amino-terminal segments of the K and L chains that are named fibrino-peptides A and B, respectively. The tetrapeptide GHRP interact with a complementary site on the L lobe of fibrin monomers and prevent polymerization. Furthermore, it has been reported that histidine-16 of the BL chain plays an important role in the association of fibrin.

Three different generations of plasminogen activators (Pas) have been introduced to the market. The first generation agents are Streptokinase and Urokinase. The second generation agents are Alteplase® and Acylated plasminogen streptokinase activator complex (APSAC). The third generation agents are Vampire bat plasminogen activator (BatPA), Reteplase®, Tenecteplase®, Lanatoplase®, and Staphylokinase®.

The limited fibrin specificity of tPA has prompted the development of plasminogen activators (PAs) with greater selectivity for fibrin. Thrombolytic therapy has been shown to significantly improve survival following AMI. The most common thrombolytic agents are Alteplase® (tissue-type plasminogen activator, tPA) Reteplase®, Tenecteplase®, and Lantoplase®.

Despite all progress made, current thrombolytic therapy is still associated with significant drawbacks including the need for large therapeutic doses, short half-life of the agent due to interaction with plasminogen activator inhibitor-1 (PAI-1), limited fibrin specificity and the risk of either severe bleeding complications or reocclusion.

Resistance to PAI-1 is another factor which confers clinical benefits in thrombolytic therapy. The only US FDA approved PAI-1 resistant drug is Tenecteplase®. Deletion variants of t-PA have the advantage of fewer disulfide bonds in addition to higher plasma half lives.

Development of various forms of t-PA (e.g. Alteplase®, Reteplase® and Tenecteplase®) has exploited the activity of t-PA. Since the recognition that residues 296-304 are critical for the interaction of t-PA with PAI-1, several variants of t-PA with mutations or deletions in this domain have been investigated. Tenecteplase® is the only FDA approved PAI-1 resistant thrombolytic agent. Tenecteplase® consists of two point mutations at positions 103, 117 that causes prolonged plasma half life. Furthermore, the four amino acids at position 296-299 have been replaced by four alanines which make resistance towards inhibition by PAI-1. Reteplase® is a single-chain non-glycosylated deletion variant of t-PA consisting of only the second kringle and the protease domains. Since finger domain is the responsible domain for fibrin affinity, Reteplase® is characterized by reduced fibrin selectivity and causes more fibrinogen depletion than the full length forms. In the absence of fibrin, Reteplase and Alteplase do not differ with respect to their activity as plasminogen activators, nor do they differ in terms of their inhibition by the PAI-1.

One of the advantages of t-PA is being specific to fibrin much more than past generations creating unwanted hemorrhages. The t-PA still carries on many concerns like unspecific binding to fibrinogen and fibrin degradation products resulting in unfavourable properties threatening the life of compromised patients. Furthermore, low half-life of t-PA (5 minutes) is also problematic so that its administration should be repeated in patients. Type-1 plasminogen activator should be repeated in the patients. Type-1 plasminogen activator inhibitor (PAI-1) is the enzyme that could digest t-PA in preliminary part of protease domain reducing half-life. Thus many researchers are trying to make different version of such molecules persuading changes like prolonging half-life and altering structural features improving pharmacodynamic properties to overcome clinical usage bottlenecks. Tenectoplase is one of the drugs undergoing mutations in first part of protease domain making it resistant to PAI-1 prolonging half-life to 18 minutes. Desmoteplase is a new version of plasminogen activator is also called as DSPAalpha1 found in the saliva of vampire bat Demodus rotundus. This novel molecule owns several improved properties for usage in acute ischemic stroke treatment over the current therapy and is now under clinical trial phase III. Natural modifications in Desmoteplase make it a good candidate to be replaced for t-PA. It should be bear in mind that the main unfavorable properties of t-PA are derived from finger and kringle 2 domains. When t-PA acts on fibrinogen, the finger domain and kringle 2 domains bind to fibrinogen and fibrin degradation products in addition to fibrin degradation products in addition to fibrin through their specific binding sites.

The two domains have been subject of alteration naturally so that the sequence of fibrin has been changed as well as the whole kringle 2 domain also is removed. The modified sequence of finger domain specifically binds to fibrin but not to other compounds. The activity of Desmoteplase dramatically reduces in absence of fibrin. Further another benefit of desmoteplase is its resistance to PAI-1 enzyme prolonging half life to 2.6 hours. Despite of lack of report regarding to immunogenic reactions related to its animal origin, there may be side effects in future.

Hence there is a need to develop a variant of tissue plasminogen activator (t-pa) that has more fibrin activity and is resistant to plasminogen activator inhibitor-1.

The above mentioned shortcomings, disadvantages and problems are addressed herein and which will be understood by reading and studying the following specification.

### OBJECTIVES OF THE EMBODIMENTS

The primary objective of the embodiment herein is to provide a variant of tissue plasminogen activator (t-PA) which is resistant to PAI-1 enzyme.

Another object of the embodiment herein is to provide a novel variant of tissue plasminogen activator which has greater fibrin binding affinity when compared to the wild type t-PA.

Yet another object of the embodiment herein is to provide a variant of plasminogen activator that doesn't cause depletion of fibrinogen.

Yet another objective of the embodiment herein is to provide plasminogen activator with improved pharmacodynamic properties.

Yet another objective of the embodiment herein is to provide the chimeric t-PA similar to desmoteplase structurally but conserving the human sequence of tissue plasminogen activator as much as possible.

Yet another objective of the embodiment herein is to provide a variant of chimeric truncated t-PA with improved properties of desmoteplase without causing immunogenic reactions.

Yet another objective of the embodiment herein is to provide the mutant variant of tissue plasminogen activator having a fibrin affinity of 1.2 fold compared to native full lengths t-PA.

Yet another objective of the embodiment herein is to express the mutant variant of tissue plasminogen activator in the Pichia pastoris.

Yet another objective of the embodiment herein is to investigate and optimize a mixed-feeding strategy based on maintaining a constant specific growth rate of P. pastoris on glycerol and methanol to achieve the highest expression of CT-b

The embodiment herein will become readily apparent from the following detailed description taken in conjunction with the accompanying drawings.

### SUMMARY

The various embodiments herein provide a novel chimeric truncated form of tissue plasminogen activator (t-PA). The chimeric t-PA consists of a desmoteplase, dinger domain, followed by human finger domain, kringle 1 domain and protease domain with four alanine (AAAA) (SEQ ID NO. 1). The chimeric truncated form of tissue plasminogen activator (t-PA) is expressed in Pichia pastoris cells. The human t-PA finger domain is replaced with the finger domain of desmoteplase tissue plasminogen activator. Further the kringle 2 domain is removed and the gap sequences between kringle 1 and kringle 2 domains are maintained. The protease domain of the human t-PA is also maintained. The four alanine on the upstream of protease domain are substituted with KHRR (SEQ ID NO. 2). The obtained chimeric t-PA or CT-b has prolonged half life and increased fibrin affinity. The elevated half-life is related to kringle 2 domain deletion and replacement of desmoteplase finger domain with t-PA. The increased half life is the consequence of four alanine (AAAA) (SEQ ID NO. 1) substitutions with KHRR (SEQ ID NO. 2) making t-PA resistant to PAI enzyme.

According to one embodiment herein, a chimeric truncated tissue plasminogen activator CT t-PA or CT-b comprises a native human t-PA with an EGF domain, a kringle 1 (K1) domain and a protease domain. The finger (F) domain of native human t-PA is replaced by F domain of a desmoteplase. The kringle 2 (K2) domain of native human t-PA is removed, and the amino acids at a position of 214 to 218 are substituted. The substituted amino acids are AAAA (SEQ ID NO. 1) replaced by KHRR (SEQ ID NO. 2).

According to one embodiment herein, the chimeric truncated tissue plasminogen activator CT t-PA or CT-b has 445 amino acids. Further the chimeric truncated tissue plasminogen activator CT t-PA or CT-b has a fibrin affinity of 60%. The chimeric truncated tissue plasminogen activator CT t-PA or CT-b has a specific activity of 1136.6 IU/μg in a 2 liter fermenter.

According to one embodiment herein, the chimeric truncated tissue plasminogen activator CT t-PA or CT-b has a residual activity of 90% after exposure to plasminogen activator inhibitor-1 (PAI-1). Also the chimeric truncated tissue plasminogen activator CT t-PA or CT-b has an amidolytic activity in a range of 46 to 83 IU/ml.

According to one embodiment herein, the chimeric truncated tissue plasminogen activator CT t-PA or CT-b has a catalytic activity and wherein the catalytic activity of the t-PA is increased by 1560 times in presence of fibrin. The chimeric truncated tissue plasminogen activator CT t-PA or CT-b has a molecular weight in the range of 52 kDa-77 kDa. Also the chimeric truncated tissue plasminogen activator CT t-PA or CT-b has a fibrin affinity of 1.2 times higher than a fibrin affinity of normal t-PA having a full length.

According to one embodiment herein, the achieved specific activity of t-PA or CT-b in 2 liter fermenter is 1136.6 IU/μg and the left enzyme activity after exposed to PAI enzyme is 90% considering that in clinical condition the high level of PAI-1 makes the thrombolytic system prone to re-occulation. The mutant version of t-PA or CT-b consists of 445 amino acids and has a fibrin affinity of 60%. The fibrin specificity or the catalytic activity of t-PA or CT-b is 1560 times more in the presence of fibrin while no appreciate activity is observed when fibrin is absent. Further CT-b exhibits 1.2 folds higher resistance to PAI-1 enzyme. Since the kringle 2 domain is considered as one of the binding sites for PAI-1, the deletion of kringle 2 domain is considered as one of the binding sites for PAI-1, its deletion along with amino acid substitution in pro-tease domain contributes to prolonged half-life. The 90% of the CT-b molecular activity is intact after exposing to PAI-1, when compared to normal t-PA. The normal t-PA has the molecular activity of 56%. The CT-b is inhibited 44% less than the normal t-PA by PAI-1 enzyme. This demonstrates improved half life. Also the amino acids are removed at position 214 to 218. The AAAA (SEQ ID NO. 1) amino acids are substituted for KHRR (SEQ ID NO. 2).

According to one embodiment herein, the gene synthesis and codon optimization of the chimeric truncated tissue plasminogen activator (t-PA) obtained from finger domain of b-PA and human t-PA (growth, kringle 1 and protease domains) as well as amino acid substitution, AAAA (SEQ ID NO. 1) to KHRR (SEQ ID NO. 2) at positions 294-298 is performed. The gene received in pGH vector with Xho 1 and Xba 1 restriction sites. The pGH and pPICZAα vectors are amplified through transfection to top 10F′. Further both vectors are digested by Xho1 and Xba1. The vectors are then ligated by ligase at 4° C. for overnight.

After transformation of ligated mixtures to top 10F′, the matrix preparation is performed and colony selection is done with forward primers (5′ GTTGCCTGCAAGGATGAGATCACACAAATG-3′) (SEQ ID NO. 3) and reverse primers (5′TGGTCTCATGTTATCTCTGATCCAGTCCAAATA-3′) (SEQ ID NO. 4). Several clones are selected and cultured in LB-LS broth medium. A confirmatory digestion with selected restriction enzyme is performed.

According to one embodiment herein, after confirming the proper sequence arrangement by bidirectional sequencing, the clone 4 is amplified in LB-LS medium containing 100 μg/ml Zeocin™. After plasmid extraction, 10 μg of recombinant plasmid pPICZAα is linearized with SacI enzyme. The mixture is then cleaned up to remove buffer salts and then electroporated into Pichia pastoris according to invitrogen protocol. The preparation of electrocompetent Pichia strain GS115 is done as per supplier's instructions (Invitrogen™). After mixing 5-10 μg of linearized plasmid with 80 μl of electrocompetent cells, the mixture is incubated on ice for 5 min in a 0.2 cm electroporation cuvette. The cuvetted is then electroporated in a Biorad-GenePulser with settings of 1500V, 25 uF capacitance, and 400 ohms resistance. Following pulsing, 1.0 ml of ice cold 1 M sorbitol is added to the cuvette, and the cells transferred to a sterile 15 ml culture tube. The tube is incubated at 30° C. without shaking for 1 h, then 1.0 ml YPD medium was added to the tube, and the cells are allowed to recover for 2 h at 30° C. at 250 RPM. Transformants are plated (200 ml) on YPDS plates containing 200, 500 and 1000 μg/ml Zeocin™ and grown at 30° C. to isolate Zeocin-resistant transformants. A rapid colony PCR is used to confirm the presence of the CT t-PA coding sequence in transformed cells.

According to one embodiment herein, for the small scale expression in Pichia some selected colonies grown in Zeocin™ plate are first grown overnight in YPD medium containing (1% yeast extract, 2% peptone, 100 mM potassium phosphate, pH 6.0, 1.34% YNB, 4×10-5% biotin 1% glycerol) to stationary phase. Next day, the optical density is measured, and 1.0 OD600 units of each culture are suspended in 10 mls of BMGY medium and grown overnight. On the third day, the optical density is measured, and 10 OD600 units of each culture are pelleted for 30 seconds at 2000×g at room temperature. The cells are suspended in 10 ml of BMMY medium (1% yeast extract, 2% peptone, 100 mM potassium phosphate, pH 6.0, 1.34% YNB, 4×10-5% biotin, 0.5% methanol). The cultures were induced for 72 hours at 30° C. with shaking (225 rpm), adding methanol every 24 hours to substitute lost/metabolized methanol. Then, the cells are centrifuged 10,000×g for 5 minutes to separate cells from extracellular supernatants. The supernatants are transferred to a new microcentrifuge tube. Pellets and supernatants are immediately frozen and stored at −80° C. For SDS PAGE and western blotting, the best clone in terms of activity is chosen and expressed in 300 ml media culture in above condition but for 4 days.

According to one embodiment herein, for the analysis of CT t-PA analysis the activity measurement is done according to Stephen and its colleagues. In brief, t-PA or CT-t-PA with plasminogen (40 μg/ml) and plasmin substrate (0.4 mM) were incubated at room temperature. After 1 hour the absorbance is read by spectrophotometer at 405 nm. Standard curve is plotted with single chain t-PA standard diluting the standard solution (40 IU/ml) 1:4 with Assay Diluent to produce 10, 2.5, 0.625, 0.156, and 0.039 IU/ml and consequent activity is measured. Soluble fibrin is prepared according to standard known procedures i.e. (80 μg/ml) is added when needed.

According to one embodiment herein, for the purification Ni-NTA purification column (Amersham-Pharmacia Quarry Bay) is used for CT t-PA purification. To start purification a binding buffer containing 10 mM Na₂HPO₄, 300 mM NaCl, and 10 mM imidazole is prepared followed by preparing a washing buffer containing 10 mM Na₂HPO₄, 300 mM NaCl, and 20 mM imidazole. Then, elution is done using 10 mM Na₂HPO₄, 300 mM NaCl, and 400 mM imidazole at pH 8.8 according to manufacturer instruction. The potency is calculated by dividing the activity by concentration of chimeric-truncated t-PA in supernatant determined by bradford protein assay.

According to one embodiment herein, the sodium dodecyl sulfate-poly-acrylamide gel electrophoresis (SDS-PAGE) is done under reducing conditions with 12% resolving gel and 5% stacking gel. The Western blot analysis of culture media is carried out according to Sambrook et al. electro blotting is performed in semi-dry blotting system. Proteins are transferred to a nitrocellulose membrane and antigen-antibody complexes are visualized by DAB-HRP system. The primary polyclonal rabbit anti-human t-PA antibody (Abcam USA) is diluted in a 1/500 dilution and goat anti-rabbit antibody (Santa Cruz. USA) is used in a 1/1000 dilution as the secondary antibody.

According to one embodiment herein, for the endoglycosidase digestion of glycoproteins 1 mg/ml solution of denatured glycoprotein Is prepared by adding 50 mg of RNase B to 45 ml of 20 mM ammonium bicarbonate, pH 8, and then adding 5 ml of denaturation solution (0.2% SDS with 100 mM 2-mercaptoethanol). The next step is heating the solution to 100° C. for 10 minutes, which denature the glycoprotein. After allowing the solution to be cooled, 2-10 ml of the prepared PNGase F enzyme solution (500 units/ml) is added to the reaction mixture and incubated at 37° C. for 1-3 hours. After 5 minutes the reaction is stopped by heating to 100° C. Then 5-10 ml aliquot is removed to assess deglycosylation by SDS-PAGE. N-terminal analysis is done to confirm the correct protease process of signal peptides.

According to one embodiment herein, for the fibrin binding assay the binding of CT-t-PA is assessed by previously reported methods. For fibrin construction, bovine thrombin (0.5u/ml) in buffer (0.05 M Tric-HCl, pH 7.4, 0.12 M NaCl. 0.01% Tween 80, 1 mg/ml bovine serum albumin) is mixed with different concentration of fibrinogen (0-0.3 mg/ml) and incubated for 30 min at 37° C. Then, CT-t-PA or full length t-PA is added in equal units (3000) and is again incubated for 30 min at 37° C. For clot removing, centrifugation (15 min, 13000 rpm, 4 C; sigma 202 MD) is performed. Now, the amount of enzyme bound to fibrin is calculated from the difference of the total amount of enzyme and free enzyme in the supernatant, as determined by ELISA. Finally, according to Assaypro kit procedure, the residual chimeric-truncated t-PA activity is measured.

According to one embodiment herein, resistance of chimeric truncated to inhibition by PAI-1 is assessed by previously reported methods. CT-t-PA (3000 IU/ml) is incubated with different concentration of Human rPAI-1(0 to 128 μM) at 25° C. for 1 hour. Now, the residual activity was measured and compared with standard t-PA.

According to one embodiment herein, for fermentation the inoculum preparation was performed in YPD media (Glucose 20 g/L, Peptone 20 g/L, Yeast extract 10 g/L) and before transfer into fermenter, defined media culture containing glycerol (40 g/L), CaSO4 (0.9 g/1), K2SO4 (14.67 g/L), MgSO4.7H2O (11.67 g/L), (NH4)2SO4 (9 g/L), Hexametaphosphate (150 g/L), PTM1 10 (ml/l), pH 6.0 was added. The PTM1 solution contained 6.00 g CuSO4.5H2O, 0.08 g NaI, 3.00 g MnSO4.H2O, 0.20 g Na2MoO4.2H2O, 0.02 g H3BO3, 0.92 g CoCl2.6H2O, 20.00 g ZnCl2, 65.00 g FeSO4.7H2O, 0.20 g Biotin and 5.00 ml of concentrated H2SO4 per liter. The glycerol feed solution contained 40 g/L glycerol, and 1.2 ml PTM1 per liter. It is sterilized by filtration. The methanol feed solution contained 260 g methanol and 12 ml PTM1 per liter according to nature protocol.

According to one embodiment herein, in every run, from defined intervals, sample is taken and the cell density is measured according to the following equation: Biomass dry weight (g/l)=OD600×dilution factor×0.21 Methanol concentration during all experiments is measured through methanol electrode. The pH and antifoam is monitored through their attributed probes during all fermentation process. The pH probe is calibrated by standard 4 and 7 solutions.

According to one embodiment herein, the carbon source in induction phase plays an important role as supply of energy and protein synthesis. Mixed glycerol and methanol in different experiments have shown to improve expression. But the amount and ratio of consumption according to specific growth of Pichia pastoris on both methanol and glycerol is critical in expression. The experimental design software and specific growth rate on methanol and glycerol was considered as the main criteria of investigation.

Glycerol and methanol with two levels are designed employing response surface methodology (RSM) using central composite design (CCD) to scrutinize their interactive role on enzyme activity, amount of expressed protein and productivity. Central composite mode is chosen to address our two factor assessment each varied at two levels, coded as −1 (lowest value) and +1 (highest value). Totally 12 experiment run including four replicate center point are performed.

According to one embodiment herein, fed batch fermentation is performed in 5 L fermenter (Bio flow 3000) while foaming formation, pH, methanol and dissolved oxygen is monitored online. Working volume is 2 L and the volume of inoculation is 10% of volume working. For pH controlling, ammonium sulphate 28% is employed. The pH has a sluggish change from 5 to 6 with start of induction phase. (The pH is changed from 5 to 6 gradually when induction phase commenced.) Dissolved oxygen is kept between 20 to 40% and antifoam agent is used whenever required. Temperature is also set to 26° C.

The minimum agitation was set at 500 RPM while it could rise to 1000 RPM during the fermentation period for compensating essential oxygen. Air and pure oxygen are both set on 10 L/min by flow meter and could help maintain oxygen level in normal range automatically when the maximum agitation is not sufficient. After 22-24 hours of batch phase, sudden rise in DO shows finished glycerol. Transitional phase is initiated with glycerol limited feeding from 40 ml/h to 3.4 ml/h in 4 hours. After 1 hour of glycerol limited feeding 2 ml sterile methanol per liter is added to reconcile Pichia pastoris cells to methanol. After DO sunrise again, the induction phase started with glycerol and methanol feeding with different rates exponentially to achieve the designed specific growth rates according to design of expert software explained in previous section. During this phase, samples are taken for further analysis.

According to one embodiment herein, one experiment is performed in triplicate to evaluate the reproducibility. The same experiment is continued until 28 h taking sample every 4-8 hours for enzyme activity.

The total biomass and supernatant are separated after 24 hours for all experiments by centrifuge at 3000×g for 10 minutes at 4° C. The culture supernatants are stored at 4° C.

According to one embodiment herein, for enzyme activity evaluation of chimeric truncated t-PA, a proassay kit containing the plasminogen, plasmin substrate and diluents is used. The soluble fibrin is prepared according to protocol (80 μg/ml) and added to the mixture. Measurement of activity during the induction time is done according to a standard curve plotted with human full length t-PA.

According to one embodiment herein, Ni-NTA purification column containing his tag is employed for purification. Before purification, supernatant is dialyzed against dialyzing buffer containing PBS to increase purification recovery through removing additional salts. In purification process the following solution at pH 8.8 are prepared and exploited respectively. Lysis buffer (10 mM Na2HPO4, 300 mMNaCl, and 10 mM immidazol), washing buffer (10 mM Na2HPO4, 300 mMNaCl, and 20 mM immidazol) and elution buffer (10 mM Na2HPO4, 300 mMNaCl, and 400 mM immidazol).

According to one embodiment herein, the purified fractions from different experiments are separately loaded in SDD-page gel 12% and stained with Coomassie Blue R-250. Confirmatory assay is accomplished with immunoblorting by anti-t-PA. Quantity one software is used for densitometry of SDS PAGE to measure the expressed protein CT-t-PA in comparison to total protein. For further analysis N-terminal sequencing is conducted.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The other objects, features and advantages will occur to those skilled in the art from the following description of the preferred embodiment and the accompanying drawings in which:

FIG. 1 illustrates a flowchart indicating a method for synthesizing and testing the chimeric truncated tissue plasminogen activator (CT tPA), according to an embodiment herein.

FIG. 2 illustrates an optimized codon sequence of the chimeric truncated tissue plasminogen activator (CT t-PA), according to an embodiment herein.

FIG. 3 illustrates a graph indicating amidolytic activity of chimeric truncated tissue plasminogen activator (CT t-PA), according to an embodiment herein.

FIG. 4A-4C illustrates response surface contour plots for the effect of two variables on the enzyme activity, yield and potency of chimeric truncated tissue plasminogen activator (CT t-PA), according to an embodiment herein.

FIG. 5 illustrates an image showing the Ni-NTA purification results of chimeric truncated tissue plasminogen activator (CT t-PA), according to an embodiment herein.

FIG. 6 illustrates a graph indicating enzymatic activity assay of chimeric truncated tissue plasminogen activator (CT t-PA), according to an embodiment herein.

FIG. 7A-7B illustrates images showing the SDS-PAGE and Western blot analysis of the purified protein chimeric truncated tissue plasminogen activator (CT t-PA), according to an embodiment herein.

FIG. 8 illustrates an image showing the endoglycosidase digestion of glycoproteins of chimeric truncated tissue plasminogen activator (CT t-PA), according to an embodiment herein.

FIG. 9 illustrates a graph indicating fibrin binding assay of chimeric truncated tissue plasminogen activator (CT t-PA), according to an embodiment herein.

FIG. 10 illustrates a graph indicating PAI-1 resistance assay of chimeric truncated tissue plasminogen activator (CT t-PA), according to an embodiment herein.

FIG. 11 illustrates a schematic representation of chimeric truncated tissue plasminogen activator (CT t-PA), according to one embodiment herein.

Although the specific features of the embodiments herein are shown in some drawings and not in others. This is done for convenience only as each feature may be combined with any or all of the other features in accordance with the embodiments herein.

### DETAILED DESCRIPTION OF THE EMBODIMENTS

In the following detailed description, a reference is made to the accompanying drawings that form a part hereof, and in which the specific embodiments that may be practiced is shown by way of illustration. The embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

The various embodiments herein provide a novel chimeric truncated form of tissue plasminogen activator (t-PA). The chimeric t-PA consists of a desmoteplase, dinger domain, followed by human finger domain, kringle 1 domain and protease domain with four alanine (AAAA) (SEQ ID NO. 1). The chimeric truncated form of tissue plasminogen activator (t-PA) is expressed in Pichia pastoris cells. The human t-PA finger domain is replaced with the finger domain of desmoteplase tissue plasminogen activator. Further the kringle 2 domain is removed and the gap sequences between kringle 1 and kringle 2 domains are maintained. The protease domain of the human t-PA is also maintained. The four alanine on the upstream of protease domain are substituted with KHRR (SEQ ID NO. 2). The obtained chimeric t-PA or CT-b has prolonged half life and increased fibrin affinity. The elevated half-life is related to kringle 2 domain deletion and replacement of desmoteplase finger domain with t-PA. The increased half life is the consequence of four alanine (AAAA) (SEQ ID NO. 1) substitutions with KHRR (SEQ ID NO. 2) making t-PA resistant to PAI enzyme.

According to one embodiment herein, a chimeric truncated tissue plasminogen activator CT t-PA or CT-b comprises a native human t-PA with an EGF domain, a kringle 1 (K1) domain and a protease domain. The finger (F) domain of native human t-PA is replaced by F domain of a desmoteplase. The kringle 2 (K2) domain of native human t-PA is removed, and the amino acids at a position of 214 to 218 are substituted. The substituted amino acids are AAAA (SEQ ID NO. 1) replaced by KHRR (SEQ ID NO. 2).

According to one embodiment herein, the chimeric truncated tissue plasminogen activator CT t-PA or CT-b has 445 amino acids. Further the chimeric truncated tissue plasminogen activator CT t-PA or CT-b has a fibrin affinity of 60%. The chimeric truncated tissue plasminogen activator CT t-PA or CT-b has a specific activity of 1136.6 IU/μg in a 2 liter fermenter.

According to one embodiment herein, the chimeric truncated tissue plasminogen activator CT t-PA or CT-b has a residual activity of 90% after exposure to plasminogen activator inhibitor-1 (PAI-1). Also the chimeric truncated tissue plasminogen activator CT t-PA or CT-b has an amidolytic activity in a range of 46 to 83 IU/ml.

According to one embodiment herein, the chimeric truncated tissue plasminogen activator CT t-PA or CT-b has a catalytic activity and wherein the catalytic activity of the t-PA is increased by 1560 times in presence of fibrin. The chimeric truncated tissue plasminogen activator CT t-PA or CT-b has a molecular weight in the range of 52 kDa-77 kDa. Also the chimeric truncated tissue plasminogen activator CT t-PA or CT-b has a fibrin affinity of 1.2 times higher than a fibrin affinity of normal t-PA having a full length.

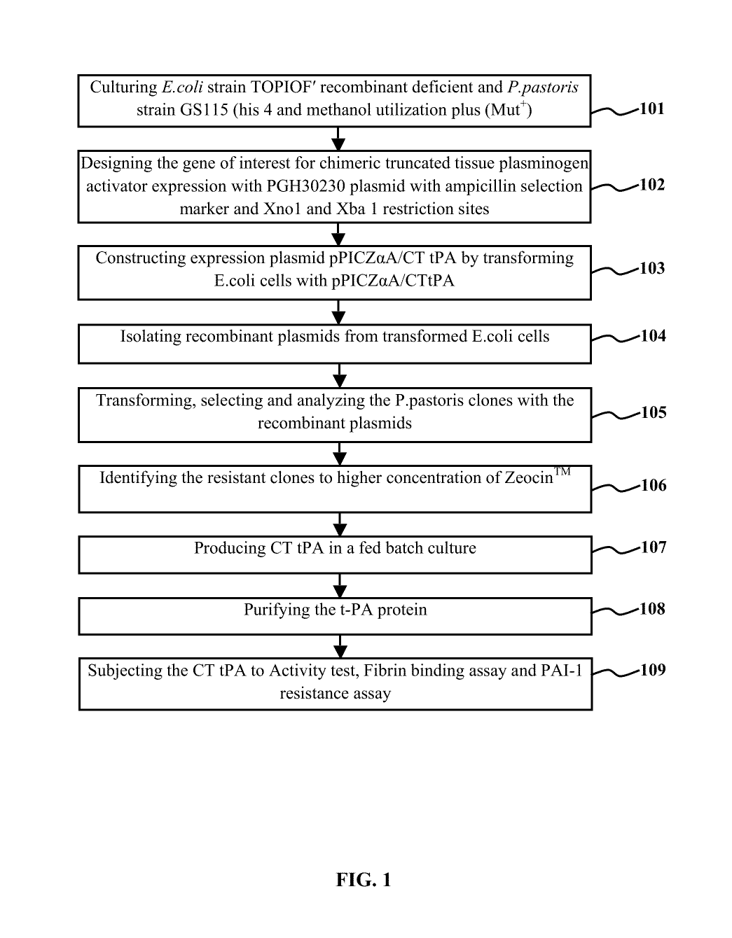

FIG. 1 illustrates a flowchart indicating a method for synthesizing and testing the chimeric truncated tissue plasminogen activator (CT tPA), according to an embodiment herein. With respect to FIG. 1, the first step is culturing E. coli strain TOP10F′ recombinant deficient and P. pastoris strain GS115 (his 4 and methanol utilization plus (Mut⁺) (101). The next step is designing the gene of interest for chimeric truncated tissue plasminogen activator expression with pGH 30230 plasmid with ampicillin selection marker and Xno1 and Xba 1 restriction sites (102). Further constructing expression plasmids pPICZαA/CT tPA by transforming E. coli cells with pPICZαA/CT tPA (103). After constructing the expression plasmids, isolating recombinant plasmids from transformed E. coli cells (104). The next step is transforming, selecting and analyzing the P. pastoris clones with the recombinant plasmids (105). Identifying the resistant clones to higher concentration of Zeocin™ (106). Producing CT t-PA in a fed batch culture (107). Purifying the CT t-PA protein (108). Subjecting CT t-PA to Activity test, Fibrin binding assay and PAI-1 Restriction assay (109).

### EXPERIMENTAL DETAILS

### Materials and Methods

Strains, Plasmids, Culture Medium, and Reagents:

Pichiapastoris (Invitrogen) strain GS115 as the expression host and pPICZαA (Invitrogen) as the expression vector were used for heterologous protein expression. Escherichia coli strain Top10F′ (Invitrogen) cells were used in standard cloning procedures. The E. coli strain TOP10F′ which is recombination deficient (recA) and deficient in endonuclease A was used for all DNA manipulations. The P. pastoris strain GS115 (his4 and methanol utilization plus (Mut^{t})) and pPICZαA (Invitrogen) were kindly provided by Pasteur Institute of Iran.

E. coli strain TOP10F′ cells were cultured in Luria-Bertani medium (LB medium; 1% (w/v) tryptone, 0.5% (w/v) yeast extract, and 1% (w/v) NaCl, pH 7.0). Antibiotics were added to LB medium at the following final concentrations: 100 μg/ml ampicillin and 25 μg/ml Zeocin™. Low-salt LB medium (1% (w/v) tryptone, 0.5% (w/v) yeast extract, and 0.5% (w/v) NaCl, pH 7.5) was used during Zeocin™ selection procedure.

The P. pastoris strain GS115 was cultured in yeast extract peptone dextrose medium (YPD; 1% (w/v) yeast extract, 2% (w/v) peptone, and 2% (w/v) dextrose), for YPDS the YPD was supplemented with 1 M sorbitol. Buffered glycerol complex medium (BMGY; 1% (w/v) yeast extract, 2% (w/v) peptone, 100 mM potassium phosphate pH 6.0, 1.34% (w/v) yeast nitrogen base, 4×10⁻⁵% (w/v) biotin, 1% (v/v) glycerol) and buffered methanol complex medium (BMMY) in which the glycerol in BMGY was replaced with 0.5% (v/v) methanol. For plates, agar was added to a final concentration of 1.5% (w/v). Cultivation of P. pastoris strains happened at 30° C. For P. pastoris, Zeocin™ was added to a final concentration of 100 μg/ml for selection of transformants.

Inoculum preparation was performed in YPD media (glucose 20 g/L, peptone 20 g/L, yeast extract 10 g/L) for 20 hours at 30° C. and 250 rpm. The fermentation medium consisted of a basal salts medium and 10 mLL⁻¹ of a trace elements solution. The fermentation medium comprises of glycerol (40 g/L), CaSO₄ (0.9 g/1), K₂SO₄ (14.67 g/L), MgSO₄.7H₂O (11.67 g/L), (NH₄)SO₄ (9 g/L) and hexametaphosphate (150 g/L). The trace elements solution, PTM1, contained 6.00 g CuSO₄.5H₂O, 0.08 g NaI, 3.00 g MnSO₄.H₂O, 0.20 g Na₂MoO.2H₂O, 0.02 g H₃BO₃, 0.92 g CoCl₂.6H₂O, 20.00 g ZnCl₂, 65.00 g FeSO4.7H₂O, 0.20 g Biotin and 5.00 mL of concentrated H₂SO₄ per liter. The glycerol feed solution contained 40 g/L glycerol and 1.2 mL PTM1 per liter and the methanol feed solution 260 g methanol and 12 mL PTM1 per liter.

Restriction enzymes, T4 DNA ligase, DNA markers, and protein markers were purchased from Fermentas®. The primary polyclonal rabbit anti-tPA antibody was purchased from Abcam and the secondary antibody, peroxidase conjugated goat antirabbit antibody was obtained from Santa Cruz.

Codon Optimization:

Codon optimization was done using proprietary software with the following parameters: 15% cut off was used for codon efficiency. Any codon below 15% was removed except for positions with strong secondary structures. The secondary structure was checked using a build in m-fold module. Internal ribosomal binding sites were removed.

Optimization Parameters:

Optimizes a variety of parameters that are critical to the efficiency of gene expression, including but not limited to: codon usage bias, GC content, CpG dinucleotides content, mRNA secondary structures, cryptic splicing sites, Premature PolyA sites, Internal Chi sites and ribosomal binding sites, Negative CpG islands, RNA instability motif (ARE), Repeat sequences (direct repeat, reverse repeat and Dyad repeat), Restriction sites that interfere with cloning).

Construction of Chimeric t-PA Expression Vector:

A novel CT-b construct comprising the finger domain of b-PA and the growth domain, kringle 1 and protease domains of human t-PA was designed. The PAI-1 interaction site (KHRR correspondent to amino acids 294-298 of CT-b) was also replaced by AAAA sequence. To express the final product with a native N terminus, a sequence containing XhoI restriction site and Kex2 recognition site was added to the 5′ end of the designed construct. The final codon construct was optimized according to the Pichia pastoris codon usage and was synthesized by Neday Fan company (Tehran, Iran).

Construction of the Expression Plasmid pPICZαa/CT tPA:

The gene coding for the new CT t-PA was synthesized in pGH-30230 plasmid. The synthesized gene construct was cloned into pPICZαA using XhoI/XbaI sites. The recombinant plasmid was confirmed through PCR, restriction mapping and bidirectional sequencing. The vector for the production of CT t-PA in P. pastoris was constructed using the pPICZαA vector as a backbone. The plasmid (pPICZαA/CT t-PA) was transformed into E. coli and selected on LB plates containing 25 μg/ml Zeocin™. Transformants were selected and verified by PCR, sequencing and digestion analysis. One positive transformant was grown in 100 ml liquid LB containing Zeocin™ (25 μg/ml) for 12 h and the recombinant plasmid (pPICZαA/CT t-PA) was isolated using a QIAquick column (Mini-Prep Kit, Qiagen) and sequenced.

The vector pPICZαA encoding a chimeric protein (termed as CT-b) consisting of human EGF, kringle 1 and protease domain of tissue plasminogen activator C-terminally fused to the finger domain of Desmoteplase. To express the final product with a native N-terminus, a sequence comprising XhoI restriction site and Kex2 recognition site was added to the 5′ end of the designed construct. The ligation mixture obtained was transformed into E. coli and single clones were screened for presence of the correct insert by PCR analysis using primers flanking the site of insertion. Positive transformants were amplified on E. coli. The vector DNA was purified by ion exchange chromatography and sequenced using an automated system.

Transformation, Selection, and Analysis of P. pastoris Clones:

About 10-20 μg of the SacI linearized pPICZ-CT-b plasmid was electroporated into Pichia pastoris according to Invitrogen™ instructions. 1 ml of YPD medium was added to the electroporated cells and the cells were allowed to recover for 2 h at 30° C. at 250 RPM. The transformants were plated on YPDS plates containing 200, 500 and 1000 mg/ml Zeocin and the zeocin-resistant transformants were isolated.

The presence of expression cassette was confirmed by colony PCR using the specific primers of CT-b [CT-bf:5 GTTGCCTGCAAGGATGAGATCACACAAATG-3 (SEQ ID NO. 3) and CT-br: 5′-TGGTCTCATGTTATCTCTGATCCAGTCCAAATA-3′ (SEQ ID NO. 3)].

Expression of the Transformed P. pastoris Clones:

Seven clones from high Zeocin™ concentration plates were grown to saturation in 10 ml BMGR or BMGY, and were placed in 50 ml tubes (2-3 days). The cells were in the range of 10-20 A600 units. The cells were harvested, the supernatant liquid was discarded, and then the pellet was re-suspended in 2 ml of BMMR or BMMY. The tube was covered with sterile gauze (cheese cloth) instead of a cap. The tube(s) were then returned to a 30 C shaker. At the end of 2-3 days, the cells were pelleted, and the supernatant assayed for product.

Fermentation:

The physiology of P. pastoris and the way it metabolizes carbon source for protein synthesis and its metabolic burden are affecting the clone. The optimization of feeding strategy in the induction phase is attracting interests as an approach for improving expression level. Among different feeding strategies used in P. pastoris fed-batch cultures, those trying to maintain a constant specific growth rate have usually resulted in superior productivities, probably because of the intrinsic connection between growth and recombinant protein production.

Invitrogen™ recommends fermentation with only methanol in the induction phase but many advantages are reported in the literature for mixed feeding strategy, like higher protein expression, shorter induction phase and increased cell viability. Moreover, the mixed feeding strategy contributes to reduced heat production and oxygen demand, which are the main bottlenecks in the large-scale production of recombinant proteins in P. pastoris. The mixed feeding strategy commonly improves productivity but uncontrolled amounts of glycerol and methanol could overturn the result. For example, excess glycerol in culture medium directly represses the AOX1 promoter and, consequently, reduces recombinant protein expression. The excess glycerol indirectly causes accumulation of acetate and ethanol in culture medium, decreasing the expression level. Over feeding of methanol leads to the accumulation of formaldehyde and ethanol as toxic components. Low methanol concentration cannot fully induce the AOX1 promoter. The finding of the fact that another source of carbon helps in enhanced amino acid synthesis and elevated expression initiated the application of different mixed feeding strategies. Nevertheless, most of the applied mixed feeding strategies are based on arbitrary ratios of the carbon sources and not considering the growth kinetics or at most the feed rate of one carbon source is considered variable while keeping another one fixed. It is investigated that the mixed feeding of methanol and glycerol on recombinant protein production in P. pastoris through a one factor at a time approach using predetermined constant specific growth rate feeding strategy and found that μGly/μMeOH=2 gives the highest expression level. It is found that amount of glycerol along with methanol is crucial in protein expression as excess glycerol could produce ethanol having negative effect on productivity in Mut-strains. This suggests limited usage of glycerol in mixed feeding strategy. Considering the lack of a comprehensive and well-designed study on the mixed glycerol-methanol feeding in fed-batch cultures of P. pastoris and its optimization thereof, the objective of the present study was to use response surface methodology (RSM) based on central composite design (CCD) to achieve an optimized mixed methanol-glycerol feeding strategy with constant specific growth rate for P. pastoris and to gain the highest expression level in a Mut+ strain genetically manipulated for the production of chimeric truncated t-PA.

Experimental Design of Fermentation and Analysis:

Glycerol and methanol specific feeding rates were designed employing Central composite based response surface methodology (CCD-based RSM) to examine their interactive role on enzyme activity, amount of expressed protein and productivity. Central composite mode was chosen to address two factor assessment each varied at two levels, coded as −1 (lowest value) and +1 (highest value). Totally, 12 fed-batch runs including four replicates for center point were performed. Design-Expert software (version 7.0, Stat-Ease Inc., MN, USA) was used to build up experimental matrix of involved factors as shown in Table 1 below:

| | | | Responses | | | |
|---|---|---|---|---|---|---|
| | Variables | | Biomass | Enzyme | Yield | |
| Experi- | μₘₑₜₕₐₙₒₗ | μ_{glycerol} | concentration | activity | (mg/ | Potency |
| ment | (h⁻¹) | (h⁻¹) | (g/L) | (IU/mL) | mL) | (IU/mg) |
| N1 | 0.03 | 0.08 | 280 | 234000 | 0.26 | 900000 |
| N2* | 0.02 | 0.05 | 280 | 341000 | 0.3 | 1136666 |
| N3 | 0.03 | 0.02 | 220 | 182000 | 0.21 | 866666 |
| N4* | 0.02 | 0.05 | 230 | 297000 | 0.27 | 1100000 |
| N5 | 0.01 | 0.02 | 240 | 311000 | 0.28 | 1110714 |
| N6 | 0.02 | 0.09 | 320 | 31000 | 0.1 | 310000 |
| N7 | 0.02 | 0.01 | 185 | 195000 | 0.23 | 847826 |
| N8 | 0.01 | 0.05 | 365 | 156801 | 0.16 | 980006 |
| N9* | 0.02 | 0.05 | 255 | 310000 | 0.33 | 939393 |
| N10* | 0.02 | 0.05 | 220 | 279000 | 0.29 | 962068 |
| N11 | 0.03 | 0.05 | 250 | 340000 | 0.3 | 1133333 |
| N12 | 0.01 | 0.08 | 400 | 11000 | 0.06 | 183333 |

The present invention investigates and optimizes a mixed-feeding strategy based on maintaining a constant specific growth rate of P. pastoris on glycerol and methanol to achieve the highest expression of CT-b. Enzyme activity, amount of protein expressed per liter and potency were to be explored in a CCD factorial study. This way, the dependency of responses to specific feeding rate of glycerol and methanol and their interactions was clarified.

Protein Purification:

The P. pastoris culture supernatant was dialyzed against PBS buffer using Medicell MWCO 12000-14000 Da dialysis bag (Medicell International Ltd., England). Ni-NTA purification column (Amersham-Pharmacia Quarry Bay) was used for CT-b purification. The binding buffer containing 10 mM Na₂HPO₄, 300 mM NaCl and 10 mM imidazole was applied to the column. The washing step was processed exploiting washing buffer comprising 10 mM Na₂HPO₄, 300 mM NaCl and 400 mM imidazole at pH 8.8 according to the manufacturer instruction.

Enzymatic Activity Assay:

Chromogenic activity kit (Assaypro, USA) was used to assess the biological activity of tissue plasminogen activator t-PA. Briefly, plasminogen (40 μg/ml) and plasmin substrate (0.4 mM) were mixed gently and then CT-b (20 μl) was added to the mixture. The whole reaction was kept at room temperature for 1 h, and the absorbance was read by spectrophotometer at 405 nm. Soluble fibrin (80 μg/ml) was added where needed. The biological activity standard curve was plotted using standard t-PA as suggested by manufacturer. All assays performed in triplicates. The apparent yellow color as absorbance was measured by spectrophotometer at 405 nm. Standard curve was plotted with single chain t-PA standard diluting the standard solution (40 IU/ml) 1:4 with assay diluent to produce 10, 2.5, 0.625, 0.156, and 0.039 IU/ml. Then, the logarithmic values of standard units were plotted against the logarithmic values of corresponded absorbance and resulting absorbance from triplicate samples was measured. Then, mean value was assayed as biological activity.

SDS-PAGE Analysis and Western Blot Analysis:

Sodium dodecyl sulfate-poly-acrylamide gel electrophoresis (SDS-PAGE) and western blot analysis were carried out according to standard methods. Protein bands were separated on SDS gel and transferred to nitrocellulose membrane using a semi-dry blotting system (Biorad, USA). A polyclonal rabbit anti-human t-PA anti-body (Abcam, USA) was used as the primary antibody and a HRP labeled goat anti-rabbit antibody (Santa Cruz, USA) was used as the secondary antibody. The antigen-antibody complexes were visualized by DAB staining. To detect protein on SDS-PAGE, clone number 10 was grown in 300 ml media culture for 96 hours, harvested and purified by Ni-NTA column. SDS-PAGE was followed by Coomassie staining.

Endoglycosidase Digestion of Glycoproteins:

After sodium dodecyl sulfate-poly-acrylamide gel electrophoresis (SDS-PAGE) and western blot analysis the mannose was removed from chimeric truncated tissue plasminogen activator t-PA (CT-b) by PNGase F enzyme. The purified CT-b as a glycoprotein was subjected to endoglycosidase assay. The protein was denatured by heating in 0.2% SDS, 100 mM2-mercaptoethanol buffer at 100° C., 10 minutes. When the solution was cooled the denatured protein was used in a PNGase digestion reaction using 2-10 ml of the prepared PNGase F enzyme solution (500 units/ml). The digestion mixture was incubated at 37° C. for 1-3 hours and the reaction was stopped by heating at 100° C. Then 5-10 ml aliquot was removed to assess deglycosylation by SDS PAGE. The digestion of CT-b with pNGase presented reduction in protein size.

Fibrin Binding Assay:

The chimeric truncated tissue plasminogen activator t-PA (CT-b) fibrin binding activity was determined. To prepare the fibrin clot the bovine thrombin (0.5μ/ml) in buffer (0.05 M Tris-HCl, pH 7.4, 0.12M NaCl, 0.01% Tween 80, 1 mg/ml bovine serum albumin) was mixed with different concentrations of fibrinogen (0-0.3 mg/ml) (Sigma-Aldrich, USA) and incubated for 30 minutes at 37° C. Then CT-b or full length t-PA was added in equal units (3000) and incubated for 30 minutes at 37° C. Centrifugation (15 minutes, 13000 rpm, 4° C., sigma 202 MD) was performed to remove existing clots. The amount of enzyme bound to fibrin was calculated from the difference of the total amount of enzyme and free enzyme in the supernatant, as determined by ELISA. Fibrin binding activity of CT-b and standard t-PA was measured in presence of different concentrations of fibrinogen.

PAI-1 Resistance Assay:

The chimeric truncated tissue plasminogen activator t-PA (CT-b) to PAI-1 enzyme was assessed according to standard protocol. Full-length t-PA and CT-b (3000 IU/ml) were incubated with different concentrations of Human PAI-1 (0 to 100 μg/ml) (Sigma-Aldrich, USA) at 25° C. for 1 hour. Then, residual activity measurement was performed by AssayPro kit. The resistance property of CT-b and the full length t-PA against PAI-1 enzyme was measured. Different concentrations of PAI-1 were added to equal amounts of CT-b and standard t-PA. The remaining biological activity of both CT-b and standard t-PA was investigated after 1 hour incubation.

### Results

Codon Optimization:

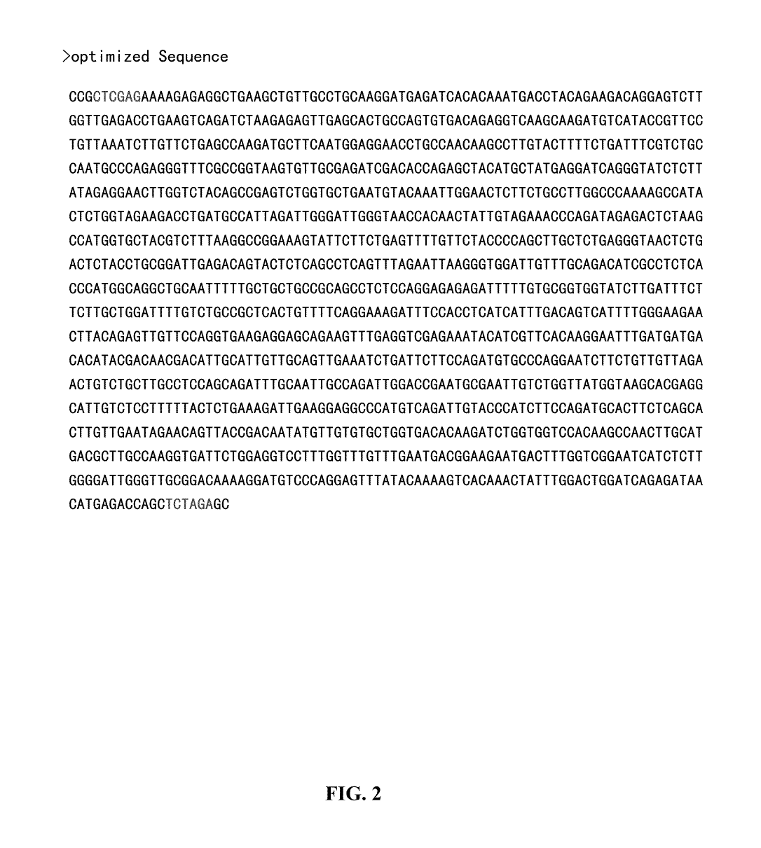

FIG. 2 illustrates an optimized codon sequence of the chimeric truncated tissue plasminogen activator (CT t-PA), according to an embodiment herein. Codon usage optimization basically involves altering the rare codons in the target gene tissue plasminogen activator (CT t-PA). The codon optimization is done so that the genes more closely reflect the codon usage of the host without modifying the amino acid sequence of the encoded protein.

Expression Analysis:

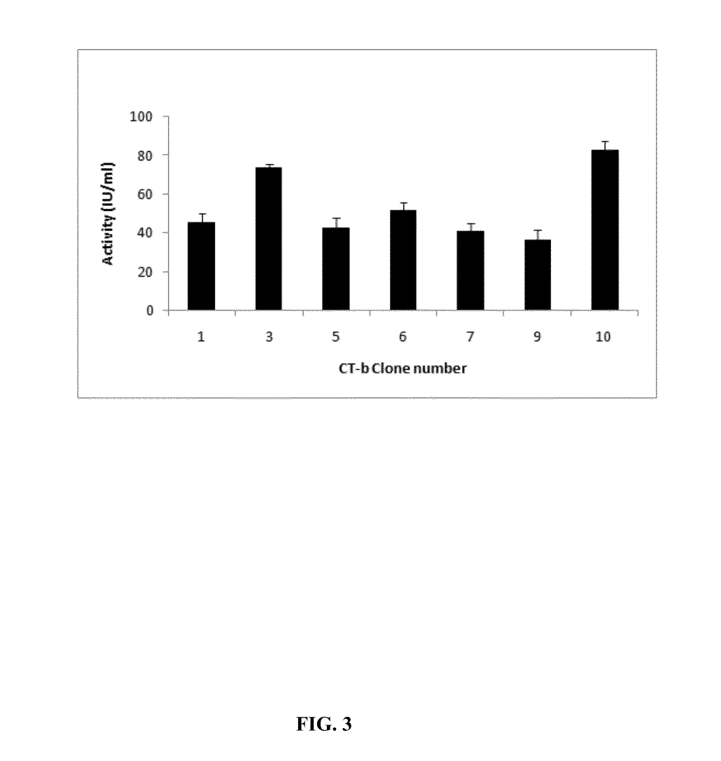

FIG. 3 illustrates a graph indicating amidolytic activity of chimeric truncated tissue plasminogen activator (CT t-PA), according to an embodiment herein. Several colonies are examined for amidolytic activity and the results shown in FIG. 3 indicate a range of expression from 46 to 83 IU/ml.

Experimental Design and Analysis for Glycerol and Methanol Specific Feeding Rates:

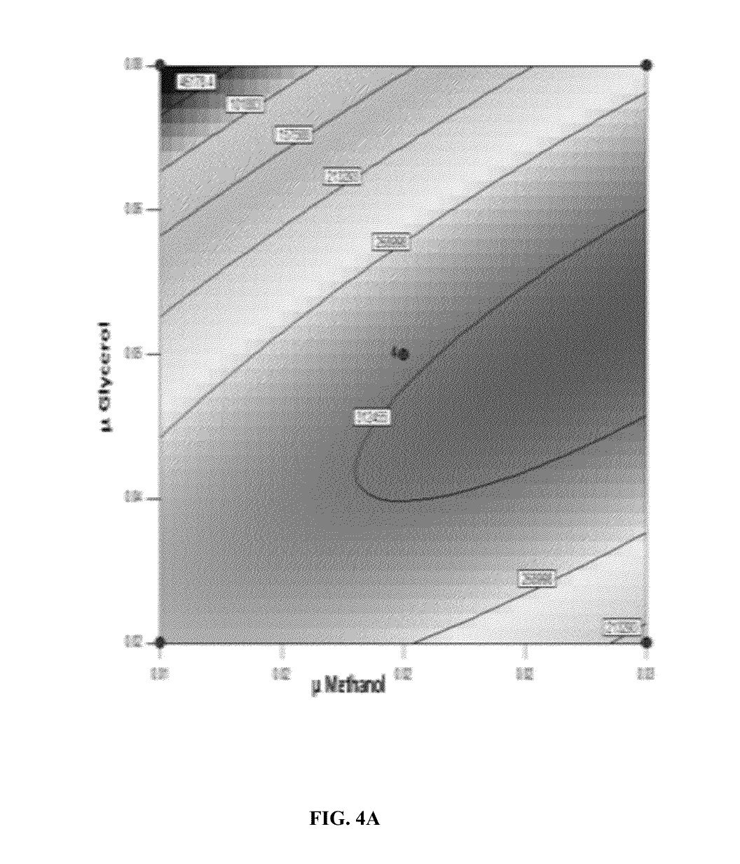

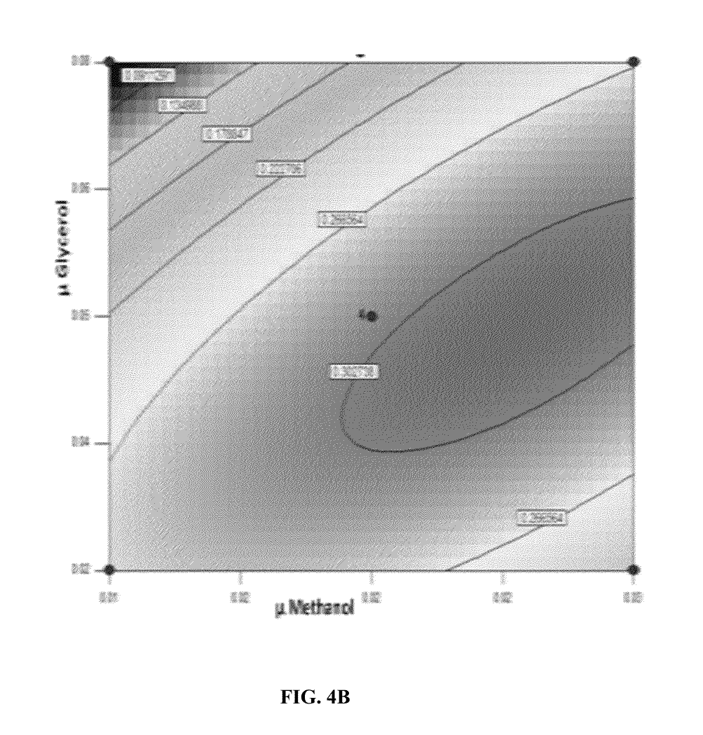

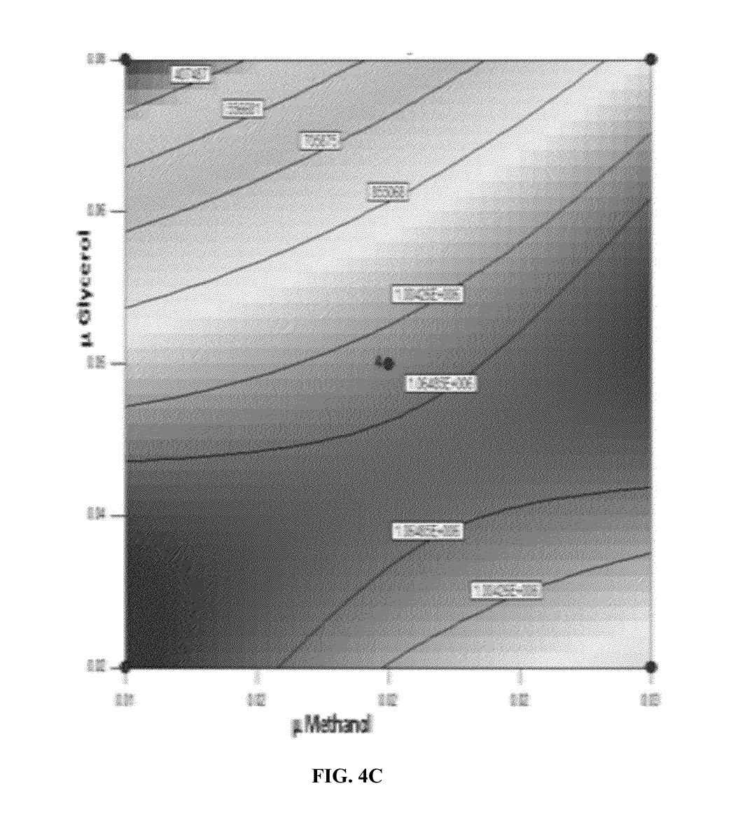

Results obtained from each experiment are provided in Table 1. According to some preliminary experiments, the highest expression levels are obtained at 24 hours. FIG. 4A-4C illustrates response surface contour plots for the effect of two variables on the enzyme activity, yield and potency of chimeric truncated tissue plasminogen activator (CT t-PA), according to an embodiment herein. According to Table 1, the highest expression or enzyme activity is achieved in experiments n2 and N11 with 340000 IU/mL while the lowest expression is associated with N12 with the enzyme activity and protein production of 11000 IU/mL and 0.06 mg/mL, respectively. For specific protein production (potency) the highest experimental results are in the range of 110000-1136666 IU/mg for experiments N2, N4, N5 and N11. The best results are achieved when the ratio of specific glycerol feeding rate to that of methanol is around.

The regression model obtained from the second order model is evaluated through the analysis of variance (ANOVA). P value and Fisher's F-test elucidate the regression significance of three responses encompassing the enzyme activity, yield and potency. Table 2 below shows the F value of the enzyme activity, yield and enzyme activity 29.3, 36.52 and 27.18 respectively. The results clarify that the model is significant. Further p-value of the response at 5% level of significance is <0.005 confirming the reliability of the model and ANOVA analysis. The chance that model F-value this large could occur due to noise is 0.04, 0.02 and 0.05 for the enzyme activity, yield and potency correspondingly.

| | | | | | |
|---|---|---|---|---|---|
| | Sum of | | Mean | F | p- |
| Source | Squares | DF | Squares | Value | value |

Biological activity (IU/mL)

| | | | | | |
|---|---|---|---|---|---|
| Model | 1.35E+11 | 5 | 2.70E+10 | 29.3 | 0.0004 |
| A- | 1.56E+10 | 1 | 1.56E+10 | 16.9 | 0.0062 |
| Methanol | | | | | |
| B- | 2.88E+10 | 1 | 2.88E+10 | 31.3 | 0.0014 |
| Glycerol | | | | | |
| AB | 3.10E+10 | 1 | 3.10E+10 | 33.6 | 0.0011 |
| A\hat{ }2 | 5.10E+09 | 1 | 5.10E+09 | 5.5 | 0.0567 |
| B\hat{ }2 | 5.89E+10 | 1 | 5.89E+10 | 64 | 0.0002 |
| Residual | 5.52E+09 | 6 | 9.19E+08 | | |
| Lack of | 3.47E+09 | 3 | 1.16E+09 | 1.6 | 0.3381 |
| Fit | | | | | |
| Pure | 2.05E+09 | 3 | 6.83E+08 | | |
| Error | | | | | |

Yield (mg/mL)

| | | | | | |
|---|---|---|---|---|---|
| Model | 0.077 | 5 | 0.015 | 36.52 | 0.0002 |
| A- | 0.013 | 1 | 0.013 | 31.93 | 0.0013 |
| Methanol | | | | | |
| B- | 0.016 | 1 | 0.016 | 37.16 | 0.0009 |
| Glycerol | | | | | |
| AB | 0.018 | 1 | 0.018 | 43.28 | 0.0006 |
| A\hat{ }2 | 6.76E−03 | 1 | 6.76E−03 | 16.05 | 0.0071 |
| B\hat{ }2 | 0.027 | 1 | 0.027 | 64.21 | 0.0002 |
| Residual | 2.53E−03 | 6 | 4.21E−04 | | |
| Lack of | 6.52E−04 | 3 | 2.17E−04 | 0.35 | 0.7957 |
| Fit | | | | | |
| Pure | 1.88E−03 | 3 | 6.25E−04 | | |
| Error | | | | | |

Potency (IU/mg)

| | | | | | |
|---|---|---|---|---|---|
| Model | 1.02E+12 | 5 | 2.04E+11 | 27.18 | 0.0005 |
| A- | 5.94E+10 | 1 | 5.94E+10 | 7.93 | 0.0305 |
| Methanol | | | | | |
| B- | 3.42E+11 | 1 | 3.42E+11 | 45.67 | 0.0005 |
| Glycerol | | | | | |
| AB | 2.31E+11 | 1 | 2.31E+11 | 30.79 | 0.0014 |
| A\hat{ }2 | 2.78E+07 | 1 | 2.78E+07 | 3.71E−03 | 0.9534 |
| B\hat{ }2 | 3.72E+11 | 1 | 3.72E+11 | 49.59 | 0.0004 |
| Residual | 4.50E+10 | 6 | 7.49E+09 | | |
| Lack of | 1.59E+10 | 3 | 5.31E+09 | 0.55 | 0.6826 |
| Fit | | | | | |
| Pure | 2.90E+10 | 3 | 9.67E+09 | | |
| Error | | | | | |

Form the p-values and considering the significant interacting terms, it is revealed that μglycerol and μmethanol both play important roles in production of the protein, the enzyme activity and potency. According to this analysis of data, the p-value of the interaction term, AB, is lower for the enzyme activity and yield in comparison to each one separately. For potency, the role of μglycerol is more significant and its p-value is lower than other parameters, even though, p-values for all of the responses are lower than 0.005 and considered significant statistically.

Equations obtained from the quadratic model for the enzyme activity, yield and potency versus μ_{glycerol} and μₘₑₜₕₐₙₒₗ are provided in terms of coded values in Equations 2, 3 and 4, respectively.

Y=3.068E+44135.31×A−59991.38×B+88000.00×A×B−28224.81×A²−95925.06×B² Equation 2

Y=+0.30+0.041×A−0.044×B+0.068×A×B−0.032×A²−0.065×B² Equation 3

Y=+1.035E+006+86182.04×A−2.068E+005×B+2.402E+005×A×B−2084.51×A²−2.410E+005×B² Equation 4

Evaluation of Enzyme Activity, Yield and Potency According to Independent Factors and their Interactive Effects:

In order to evaluate the interactive role of independent variables on the responses, contour plots are drawn. The plots for each response are described according to the two independent factors. FIG. 4A-4C illustrates response surface contour plots for the effect of two variables on the enzyme activity, yield and potency of chimeric truncated tissue plasminogen activator (CT tPA), according to an embodiment herein.

FIG. 4A illustrates that when μₘₑₜₕₐₙₒₗ is low the specific feeding rates increase the specific rate of μ_{glycerol}, leading to reduced enzyme activity. In high μₘₑₜₕₐₙₒₗ the enzyme activity rises to a maximum and then declines. In this range of μₘₑₜₕₐₙₒₗ and μ_{glycerol} the glycerol feeding enhances enzyme activity due to the higher recombinant enzyme production. Over-feeding of glycerol leads to AOX1 repression and lower enzyme activity. FIG. 4A further illustrates that the highest enzyme activity occurs in μ_{glycerol} of 0.05 h⁻¹ and μₘₑₜₕₐₙₒₗ of 0.03 h⁻¹. In other words the optimum ratio of μₘₑₜₕₐₙₒₗ and μ_{glycerol} is calculated to be 1.7.

FIG. 4B illustrates that the quantity of the expressed protein also follows the same pattern like the enzyme activity, hence the increased specific feeding rate of glycerol dramatically reduced the recombinant protein production. As shown in Table 1, the lowest yield is associated with the N12 run, which has a low methanol and a high glycerol specific feeding rate.

FIG. 4C illustrates that the potency of the enzyme. The FIG. 4C plot is obtained after dividing the enzyme activity (IU/mL) by the enzyme yield (mg/mL). The result obtained after dividing show the potency of the recombinant product. When compared to other responses, the enzyme potency showed different behavior toward the variables. Still the optimum ratio of around 1.7 is also applicable to the maximum potency. The N5 run (Table 1) and high specific growth rate, like the N11 run are among the ones displaying high potency (1,133, 333 unit/mg). This potency (specific activity) is much higher than for what is reported formerly in E. coli related to reteplase 566,917 unit/mg. This specific activity is also higher to what is reported for full length t-PA expressed in mammalian cells as 5, 80,000 unit/mg. Duteplase is another plasminogen activator expressed in yeast with 300,000 unit/mg showing less specific activity than present invention.

From FIG. 4C it is further inferred that the contour plot for the potency, the activity versus mg of the recombinant protein, that a certain ratio of the specific glycerol feeding rate to that of methanol, in any specific growth rate, leads to the maximum protein production and activity. The shaded zone in this plot corresponds to higher protein activity. The alignment of the shaded zone along a linear strip with a constant slope is a consequence of the maximum protein activity in all feeding rates is resulted from a certain optimum ratio of the specific glycerol feeding rate to methanol. With mixed feeding of methanol and glycerol in this optimum ratio, any decrease or increase in the feeding of the carbon sources changes the optimum ratio of the mixed feeding rate and accordingly decreases the protein activity. When the analysis is done at cellular level, each yeast cell i.e. P. pastoris makes the recombinant product having the maximum activity with consuming methanol and glycerol in a certain optimum ratio. Methanol and glycerol input fluxes are always maintaining a certain ratio in order to enforce balanced mixed carbon consumption toward maximum recombinant protein activity. The results show that the implemented CCD-RSM has an optimum ratio of 1.7.

In the experiment with conditions of the center point, the enzyme activity is measured up to 30 hours and the maximum expression is achieved around 24 hour. This is the consequence of several reasons. The first reason is that the tissue plasminogen activator is an enzyme possessing a protease domain responsible for self-enzyme cleavage leading to dropping activity. On the other hand this activity reduction is the result of increased biomass concentrations at late induction phase and increased tensions from the metabolic burden. Therefore most of the cellular energy is allocated to the cellular maintenance than recombinant protein production. Third reason is, in high cell density the lysis of cells leads to the release of intracellular proteases to the medium and as a result recombinant protein degradation. The stability and activity of tissue plasminogen activators are limited to their auto cleavage activity, such that after a specific period leads to reduced productivity. After 24 hours achieving the maximum wet cell weight makes it difficult to control increased temperature whereas coping with high oxygen demand is difficult.

Protein Purification:

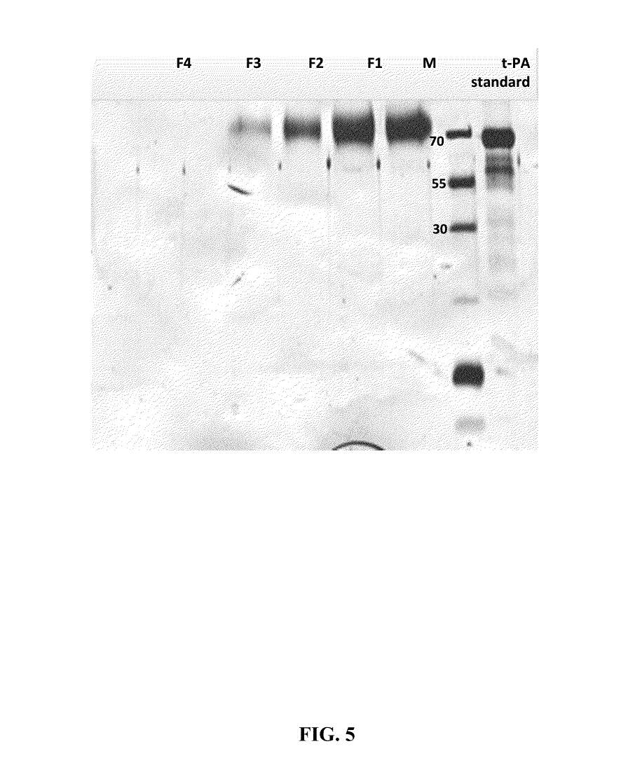

FIG. 5 illustrates an image showing the Ni-NTA purification results of chimeric truncated tissue plasminogen activator (CT tPA), according to an embodiment herein. The lane 1 of the protein purification gel is loaded with standard t-PA (positive control). The lane 2 of the protein purification gel is loaded with protein marker. The protein marker shows three bands with molecular weight of 70 kDa, 55 kDa and 30 kDa respectively. The lane 3 to lane 6 of the protein purification gel is loaded with the protein sample to be purified. FIG. 5 exhibits that the protein to be purified has a molecular weight more than or equal to 70 kDa.

Enzymatic Activity Assay:

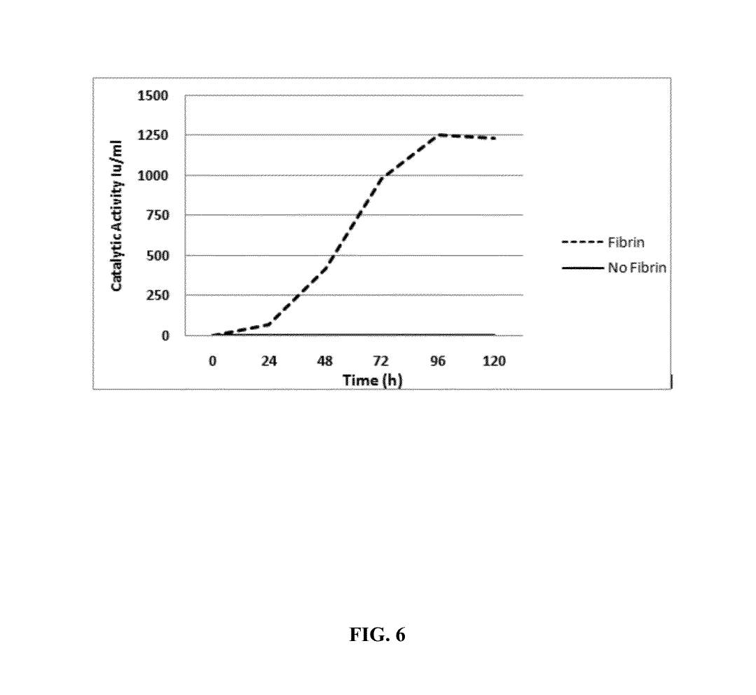

FIG. 6 illustrates a graph indicating enzymatic activity assay of chimeric truncated tissue plasminogen activator (CT t-PA), according to an embodiment herein. The expression level of the hyproducer colony (83 IU/ml) is monitored for five days in 300 ml media culture. The maximum level of 1250 IU/ml is detected after 96 hours in the presence of fibrin. No appreciated activity is observed in the absence of fibrin. The amidolytic activity is more pronounced after addition of fibrin to above mixture. In mixtures without fibrin, no measurable amidolytic activity is observed. Hence it is inferred that the higher catalytic activity of the chimeric truncated tissue plasminogen activator (CT t-PA) in the presence of fibrin is 1560 times.

SDS PAGE and Western Blot Analysis:

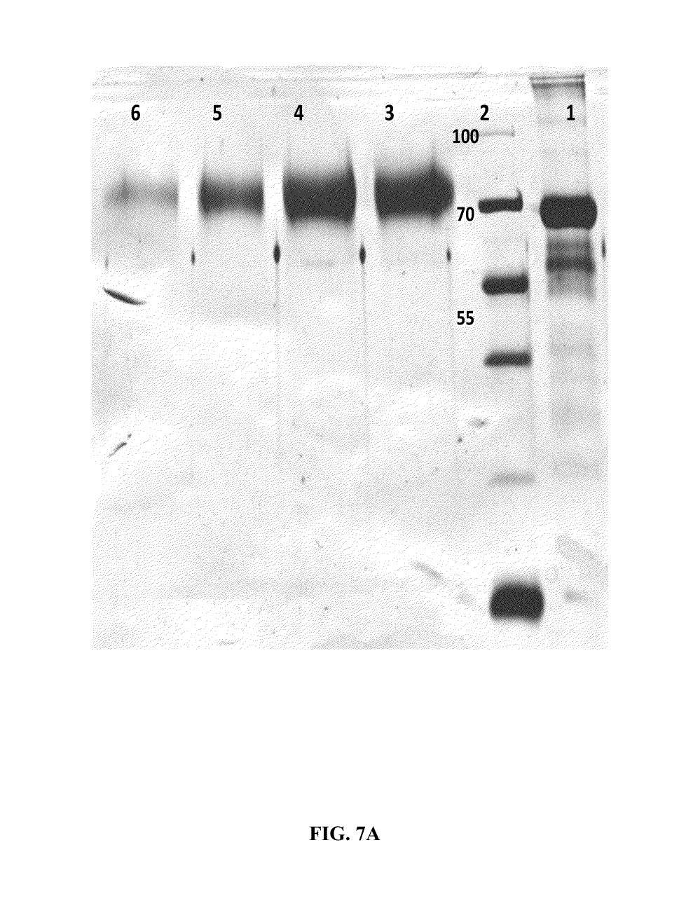

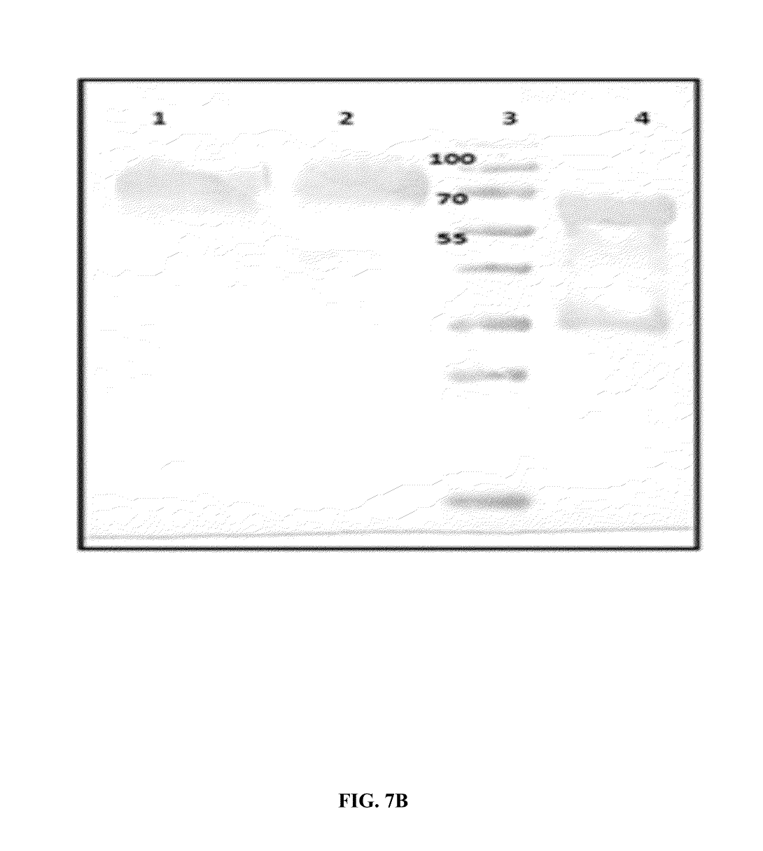

FIG. 7A-7B illustrates images showing the SDS-PAGE and Western blot analysis of the purified protein chimeric truncated tissue plasminogen activator (CT t-PA), according to an embodiment herein. FIG. 7A illustrates the SDS-PAGE results. The SDS-PAGE gel lane 1 is loaded with positive control (commercial Alteplase™). The SDS-PAGE gel lane 2 is loaded with protein marker. The lanes 3-6 of the SDS-PAGE gel are loaded with fraction 1-4 of the purified protein.

FIG. 7B illustrates the Western blot results. The lane 4 of the western blot gel is loaded with positive control (Alteplase™). The lane 3 of the western blot gel is loaded with the protein marker. The lane 2 of the western blot gel is loaded with the fraction 3 of the purified protein (protein purified by Ni-NTA). The lane 1 of the western blot gel is loaded with the fraction 4 of the purified protein (protein purified by Ni-NTA).

FIG. 7A-7B further illustrates a single band of the purified protein is found to be 77 kDa. Theoretical calculation reveals that the molecular weight of purified protein i.e. chimeric truncated tissue plasminogen activator (CT t-PA) or CT-b has a molecular weight of 52 kDa.

Endoglycosidase Digestion of Glycoprotein:

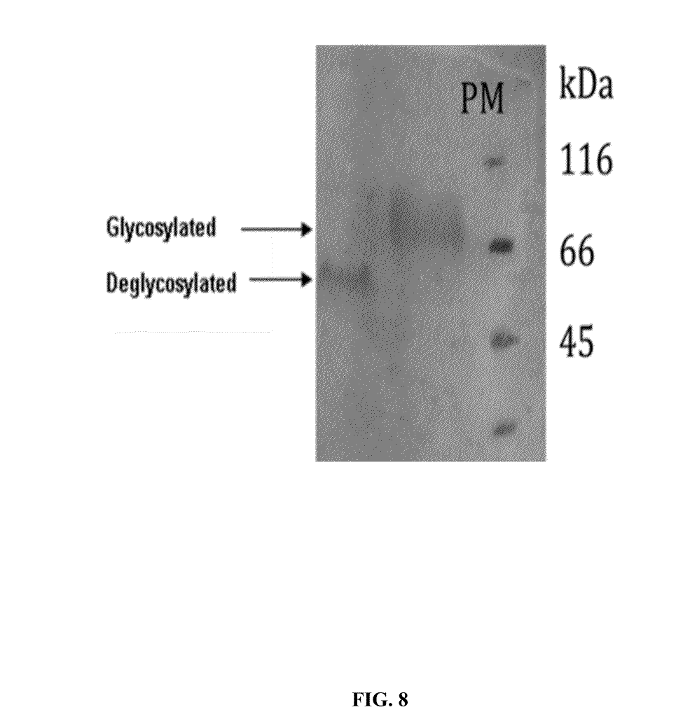

FIG. 8 illustrates an image showing the endoglycosidase digestion of glycoproteins of chimeric truncated tissue plasminogen activator (CT t-PA) or CT-b, according to an embodiment herein. The digestion of CT-b with pNGase presented a reduction in protein size from 75 kDa to 58 kDa. The N-terminal sequencing of purified protein also showed that the protein sequence is correctly separated from the signal sequence and is in correct reading frame.

Fibrin Binding Assay:

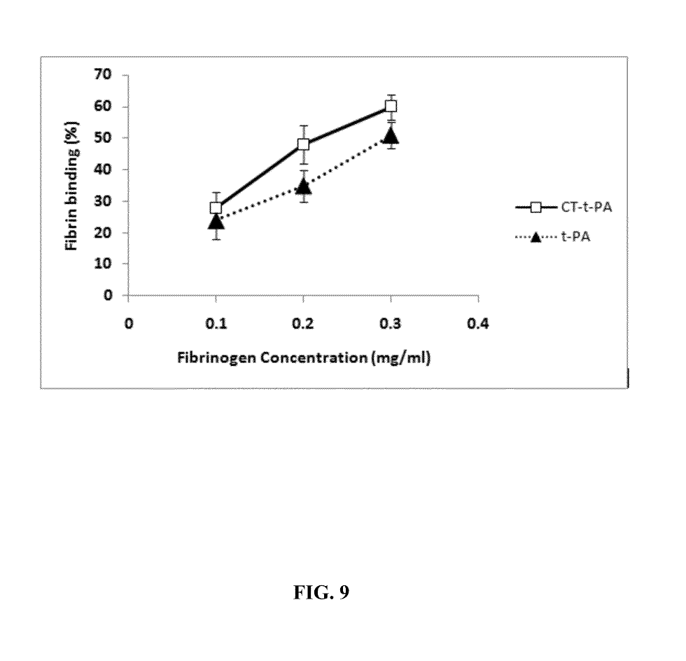

FIG. 9 illustrates a graph indicating fibrin binding assay of chimeric truncated tissue plasminogen activator (CT t-PA) or CT-b, according to an embodiment herein. FIG. 9 illustrates that the recombinant CT-b has 28% fibrin binding at a concentration of 0.1 mg/ml fibrinogen. This value of the native t-PA is calculated as 24% which is not indicating a significant difference. At a higher concentration of fibrinogen (0.3 mg/ml) the percentage of fibrin binding to CT-b and t-PA is 60% and 51% respectively. According to these data the t-PA or CT-b has 1.2 fold higher fibrin binding when compared to fill-length t-PA.

PAI-1 Resistance Assay:

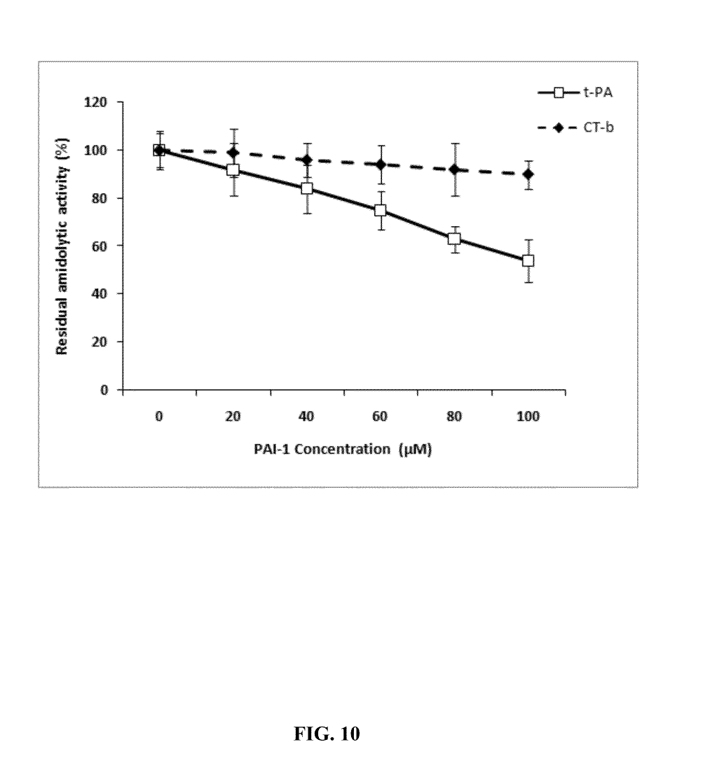

FIG. 10 illustrates a graph indicating PAI-1 resistance assay of chimeric truncated tissue plasminogen activator (CT t-PA) or CT-b, according to an embodiment herein. Full length t-PA preserve the enzyme activity up to 54% while the chimeric truncated tissue plasminogen activator (CT t-PA) or CT-b has the PAI-1 resistance of 90%. The result confirms the higher the higher resistance of recombinant CT-b to PAI-1 enzyme activity.

Structure of Chimeric Truncated Tissue Plasminogen Activator (CT t-PA) or CT-b:

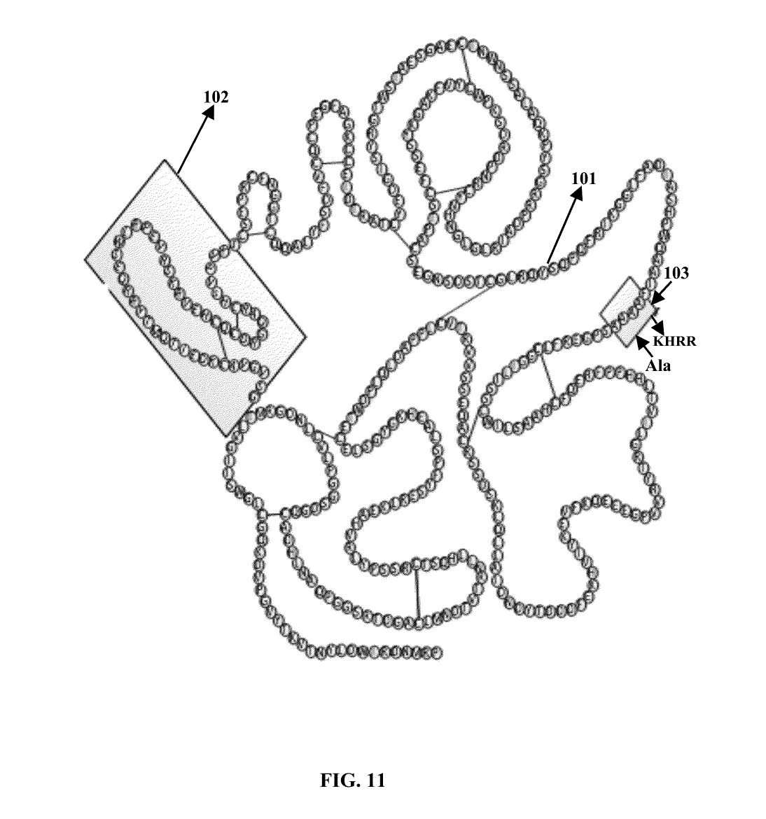

FIG. 11 illustrates a schematic representation of chimeric truncated tissue plasminogen activator (CT t-PA), according to one embodiment herein. Desmoteplase is a plasminogen activator derived from vampire bat. The animal source of desmoteplase causes immune reaction in human body. The aim of CT-b design is to conserve human sequence of human tissue plasminogen activator with desmoteplase properties. Desmoteplase holds finger domain responsible to specific binding to fibrin. The bat finger domain is replaced with human finger domain 102. Kringle 2 domain 101 is also naturally removed from its structure in comparison to human t-PA. Kringle 2 domain holds lysine binding site which binds to fibrinogen causing fibrinolysis and less specificity. In CT-b structure the kringle 2 domain is removed and desmoteplase domain is replaced for human one. The interaction site with plasminogen activator inhibitor (PAI-1) is changed from KHRR (SEQ ID NO. 2) sequence to AAAA (SEQ ID NO. 1) residues making it resistant to PAI-1 enzyme resistant and prolonged half life 103.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments.

It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

Although the embodiments herein are described with various specific embodiments, it will be obvious for a person skilled in the art to practice the invention with modifications. However, all such modifications are deemed to be within the scope of the claims.

It is also to be understood that the following claims are intended to cover all of the generic and specific features of the embodiments described herein and all the statements of the scope of the embodiments which as a matter of language might be said to fall there between.

What is claimed is:

1. A chimeric truncated tissue plasminogen activator CT t-PA or CT-b comprising:
a native human t-PA with an EGF domain, a kringle 1 (K1) domain and a protease domain,
wherein a finger (F) domain of native human t-PA is replaced by F domain of a desmoteplase, wherein the kringle 2 (K2) domain of native human t-PA is removed, wherein the amino acids at a position of 214 to 218 are substituted, wherein the substituted amino acids are AAAA (SEQ ID NO. 1) replaced by KHRR (SEQ ID NO. 2).

2. The chimeric truncated tissue plasminogen activator CT t-PA or CT-b according to claim 1, wherein the t-PA has 445 amino acids.

3. The chimeric truncated tissue plasminogen activator CT t-PA or CT-b according to claim 1, wherein the t-PA has a fibrin affinity of 60%.

4. The chimeric truncated tissue plasminogen activator CT t-PA or CT-b according to claim 1, wherein the t-PA has a specific activity of 1136.6 IU/μg in a 2 liter fermenter.

5. The chimeric truncated tissue plasminogen activator CT t-PA or CT-b according to claim 1, wherein the t-PA has a residual activity of 90% after exposure to plasminogen activator inhibitor-1 (PAI-1).

6. The chimeric truncated tissue plasminogen activator CT t-PA or CT-b according to claim 1, wherein the t-PA has an amidolytic activity in a range of 46 to 83 IU/ml.

7. The chimeric truncated tissue plasminogen activator CT t-PA or CT-b according to claim 1, wherein the t-PA has a catalytic activity and wherein the catalytic activity of the t-PA is increased by 1560 times in presence of fibrin.

8. The chimeric truncated tissue plasminogen activator CT t-PA or CT-b according to claim 1, wherein the t-PA has a molecular weight in the range of 52 kDa-77 kDa.

9. The chimeric truncated tissue plasminogen activator CT t-PA or CT-b according to claim 1, wherein the truncated t-PA has a fibrin affinity of 1.2 times higher than a fibrin affinity of normal t-PA having a full length.
